SEQUENCE LISTING (18 sequences)

SEQ ID NO: 1 - DNA, 20 bp, artificial sequence
  POU5F1 F
cttgggctcgagaaggatgt

SEQ ID NO: 2 - DNA, 20 bp, artificial sequence
  POU5F1 R
ggaaagggaccgaggagtac

SEQ ID NO: 3 - DNA, 20 bp, artificial sequence
  OTX-1 F
caagactcgctaccctgaca

SEQ ID NO: 4 - DNA, 20 bp, artificial sequence
  OTX-1 R
agtaggaagaggagggcgta

SEQ ID NO: 5 - DNA, 20 bp, artificial sequence
  CALB1 F
tggctcacgtattacccaca

SEQ ID NO: 6 - DNA, 20 bp, artificial sequence
  CALB1 R
agatccgttcggtacagctt

SEQ ID NO: 7 - DNA, 20 bp, artificial sequence
  RELN F
actttcctcctagcgctgtt

SEQ ID NO: 8 - DNA, 20 bp, artificial sequence
  RELN R
ataatcgcgccacactgttc

SEQ ID NO: 9 - DNA, 20 bp, artificial sequence
  GFAP F
agaagctccaggatgaaacc

SEQ ID NO: 10 - DNA, 20 bp, artificial sequence
  GFAP R
agcgactcaatcttcctctc

SEQ ID NO: 11 - DNA, 20 bp, artificial sequence
  CLDN11 F
ctggtggacatcctcatcct

SEQ ID NO: 12 - DNA, 20 bp, artificial sequence
  CLDN11 R
ccagcagaatgagcaaaaca

SEQ ID NO: 13 - DNA, 20 bp, artificial sequence
  Tau exon 1
gccaggagttcgaagtgatg

SEQ ID NO: 14 - DNA, 20 bp, artificial sequence
  Tau exon 2
gaggacggatctgaggaacc

SEQ ID NO: 15 - DNA, 18 bp, artificial sequence
  Tau exons 4/5
catgcgagcttgggtcac

SEQ ID NO: 16 - DNA, 18 bp, artificial sequence
  Tau exon 9
cccaagtcgccgtcttcc

SEQ ID NO: 17 - DNA, 20 bp, artificial sequence
  Tau exon 10
acttggactggacgttgcta

SEQ ID NO: 18 - DNA, 22 bp, artificial sequence
  Tau exon 11
tggtttatgatggatgttgcct

(19) United States
(12) Patent Application Publication
(10) Pub. No.: US 2022/0356445 A1
(43) Pub. Date: Nov. 10, 2022

(54) METHOD FOR IN-VITRO PRODUCTION OF MAMMALIAN NEURONS

(71) Applicants: UNIVERSITE DE BORDEAUX, BORDEAUX (FR); CENTRE NATIONAL DE LA RECHERCHE SCIENTIFIQUE, PARIS (FR); INSTITUT D'OPTIQUE THEORIQUE ET APPLIQUEE, PALAISEAU (FR); UNIVERSITE DE ROUEN-NORMANDIE, MONT SAINT AIGNAN (FR); INSTITUTE NATIONAL DE LA SANTE ET DE LA RECHERCE MEDICALE, PARIS (FR)

(72) Inventors: MAXIME FEYEUX, TALENCE (FR); KEVIN ALESSANDRI, BORDEAUX (FR); MAGALIE LECOURTOIS, HAUVILLE (FR); LAETITIA MIGUEL, LE PETIT QUEVILLY (FR); PIERRE NASSOY, BORDEAUX (FR); ANNE ROVELET LECRUX, ROUEN (FR)

(21) Appl. No.: 17/624,584

(22) PCT Filed: Jul. 3, 2020
(86) PCT No.: PCT/EP2020/068905

§ 371 Date: Jan. 4, 2022

(30) Foreign Application Priority Data
Jul. 5, 2019 (FR) FR1907554

(51) Int. Cl. C12N 5/0793

(52) U.S. Cl. CPC C12N 5/0619; C12N 2500/10; C12N 2500/32; C12N 2506/45; C12N 2533/74; C12N 2513/00

(57) ABSTRACT

The present invention relates to a method for in-vitro production of mammalian neurons expressing the 6 isoforms of the Tau protein (2N4R, 1N4R, 0N4R, 2N3R, 1N3R, 0N3R), comprising a step of neuronal differentiation, in which cellular microcompartments are cultivated for a period of 5 weeks to 100 weeks, each one comprising a hollow hydro gel capsule surrounding post-mitotic neuronal cells and an extracellular matrix, the neuronal differentiation step being carried out in a bioreactor, the cellular microcompartments being kept in suspension in an enclosure of the bioreactor containing a neuronal differentiation medium.

### CROSS-REFERENCE TO RELATED APPLICATION

This application is the U.S. national stage application of International Patent Application No. PCT/EP2020/068905, filed Jul. 3, 2020.

### REFERENCE TO SEQUENCE LISTING

The Sequence Listing for this application is labeled “Seq-List-replace.txt” which was created on Jul. 25, 2022 and is 3,502 bytes. The entire content of the sequence listing is incorporated herein by reference in its entirety.

### TECHNICAL FIELD

The invention relates to a method for the in vitro production of mammalian neurons. More particularly, the method according to the invention makes it possible to produce neurons expressing the 6 isoforms of the Tau protein (2N4R, 1N4R, 0N4R, 2N3R, 1N3R, 0N3R). The invention has applications in connection with neurodegenerative diseases, in particular in the field of diagnosis and of cell therapy, and also for the identification and development of new therapeutic treatments.

### TECHNICAL BACKGROUND

Tau is a multifunctional protein, originally identified as a microtubule-associated cytoplasmic protein. Six isoforms of Tau are expressed in the adult human brain, resulting from alternative splicing of the MAPT gene. Tau splicing is regulated during development such that only the shortest isoform of Tau is expressed in the fetal brain, unlike the adult brain which expresses the 6 isoforms.

The accumulation of pathological intracellular deposits of the Tau protein is the marker for numerous neurodegenerative diseases called tauopathies (Sergeant et al., 2008). It is important to note that the protein composition (identity of the Tau isoforms present), the morphology and the anatomical distribution of the intracellular deposits of Tau make it possible to distinguish the various tauopathies. In Alzheimer's disease (AD), the 6 Tau isoforms are abnormally phosphorylated and aggregated. In Pick's disease, the 3R Tau isoforms are predominant, whereas aggregated 4R Tau forms are prevalent in corticobasal degeneration and progressive supranuclear palsy. Furthermore, more than 50 harmful mutations have been identified within the MAPT gene in patients suffering from frontotemporal dementia linked to chromosome 17 (FTDP-17). The mutations identified result either in a reduced capacity of the Tau proteins to interact with microtubules or other partners, or in an overproduction of 4R isoforms, which leads to a change in the ratio between the 3R and 4R isoforms in the cell.

Over the past decade, human induced pluripotent stem cell (iPSC) technology has opened up new perspectives in the modeling of human diseases. Several groups have already demonstrated that iPSC-derived neurons can be a precious tool for studying tauopathies. However, the relative immaturity of iPSC-derived neurons constitutes a major challenge for the in vitro recapitulation of the Tau pathology. Numerous studies have shown that “wild-type” iPSC-derived cortical neurons express mainly the embryonic Tau isoform 0N3R (Biswas et al., 2016; Hallmann et ., 2017; Imamura et al., 2016; Iovino et al., 2015; Sato et al., 2018; Silva et al., 2016; Verheyen et al., 2018, 2015). Even though most of these studies described a transition from the single 0N3R isoform to the mixed production of 3R and 4R Tau isoforms during the differentiation procedure, the 0N3R Tau isoform remains to a large extent the predominant one. Extension of the culture time up to 365 days has allowed the detection of the 0N3R, 0N4R, 1N3R and 1N4R Tau protein isoforms in iPSC-derived neurons (Lovino et al., 2015; Sposito et al., 2015), but always with a predominance of 0N3R. The presence of adult Tau isoforms has also been described at the RNA level in iPSC-derived cortical neurons (Ehrlich et al., 2015). However, it has not been possible to detect the corresponding protein isoforms. Only the fetal Tau isoform 0N3R was present in these iPSC-derived cortical neurons.

There is therefore a need for a model of adult neurons, that is to say expressing the 6 isoforms of the T protein, in order to be able to study the tauopathies, in particular the 4R Tau-linked tauopathies.

### SUMMARY OF THE INVENTION

While working on neuron differentiation and the expression of the various isoforms of the Tau protein in adults, the inventors have discovered that it is possible to produce, in vitro, neurons expressing the 6 isoforms by culturing stem cells in alginate-based hollow cellular microcompartments, allowing a 3D culture, in a culture medium enabling neuronal differentiation. Advantageously, this step is carried out in a bioreactor in which the microcompartments are kept in suspension in said neuronal differentiation medium. The inventors have also demonstrated that, by using a particular neuronal differentiation medium comprising sodium chloride, a neuroactive inorganic salt, glycine, L-alanine and L-serine, it is possible to obtain post-mitotic neuronal cells comprising the 6 isoforms of the Tau protein in relatively short times, comprised between 5 and 50 weeks. The inventors have thus developed culture methods for obtaining such neurons after only a few weeks.

A subject of the invention is therefore a method for the in vitro production of mammalian neurons expressing the 6 isoforms of the Tau protein (2N4R, 1N4R, 0N4R, 2N3R, 1N3R, 0N3R), comprising a step of neuronal differentiation, according to which cellular microcompartments are cultured, each comprising a hollow hydrogel capsule surrounding post-mitotic neuronal cells and an extracellular matrix, said neuronal differentiation step being carried out in a bioreactor, the cellular microcompartments being kept in suspension in an enclosure of said bioreactor containing a neuronal differentiation medium for a period comprised between 5 weeks and 100 weeks.

A subject of the invention is also a method for the in vitro production of mammalian neurons expressing the 6 isoforms of the Tau protein (2N4R, 1N4R, 0N4R, 2N3R, 1N3R, 0N3R), comprising a neuronal differentiation step according to which cellular microcompartments, each comprising a hollow hydrogel capsule surrounding post-mitotic neuronal cells and an extracellular matrix in a neuronal differentiation medium comprising at least one neuroactive inorganic salt, glycine, L-alanine and L-serine, are cultured for a period comprised between 5 weeks and 100 weeks, preferentially between 5 and 50 weeks, more preferentially between 10 and 50 weeks, even more preferentially between 25 and 50 weeks, between 25 and 40 weeks, between 25 and 35 weeks, between 25 and 30 weeks, between 20 and 50 weeks, between 20 and 40 weeks, between 20 and 35 weeks, between 20 and 30 weeks, or between 20 and 25 weeks. Said compounds neuroactive inorganic salt, glycine, L-alanine and L-serine are each present at a concentration which maintains the survival and neural functionality of a neuronal cell.

A subject of the invention is also non-natural post-mitotic neuronal cells, which can be obtained by means of the method according to the invention, in which the 6 isoforms of the Tau protein are expressed.

By non-natural post-mitotic neuronal cells, it is meant cells obtained by in vitro culture under controlled conditions of cells capable of differentiating into neuronal cells, such as pluripotent cells, as opposed to cells obtained by sampling from a human subject such as an adult human subject. Thus, a subject of the invention is cell culture-derived post-mitotic neuronal cells, which can be obtained by means of the method according to the invention, in which the 6 isoforms of the Tau protein 2N4R, 1N4R, 0N4R, 2N3R, 1N3R and 0N3R are expressed.

Advantageously, such post-mitotic neuronal cells have a ratio of expression of the3R and 4R isoforms comprised between 1/3 and 3, more preferentially comprised between 1/2 and 2, even more preferentially comprised between 3/4 and 4/3, ideally at 10% of an equimolar ratio. The 2N, 1N and 0N isoforms advantageously represent, respectively, more than 3%, more than 17% and less than 90% of the total isoforms, preferentially, respectively, more than 5%, more than 26% and less than 50%, even more preferentially, respectively, more than 8%, more than 45% and less than 45%, ideally, respectively, 9%, 54%, and 37%.

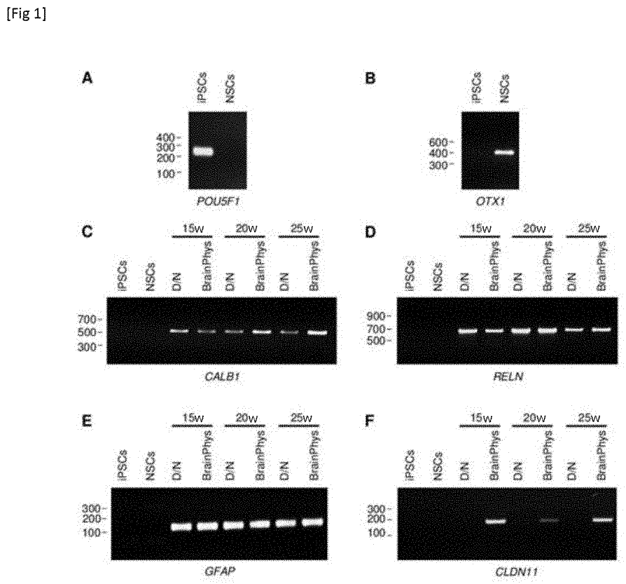

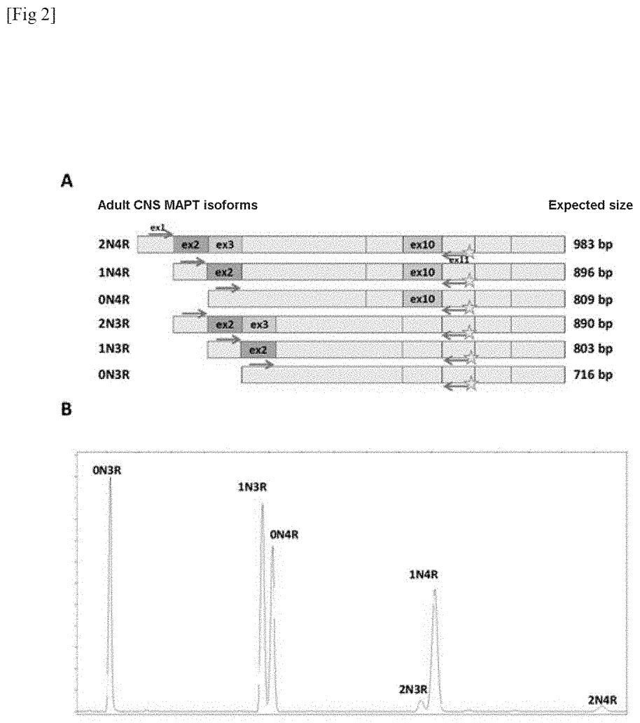

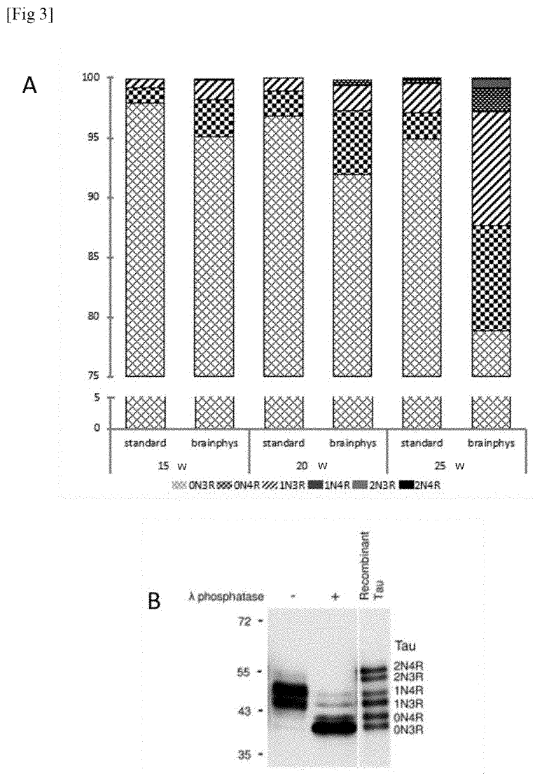

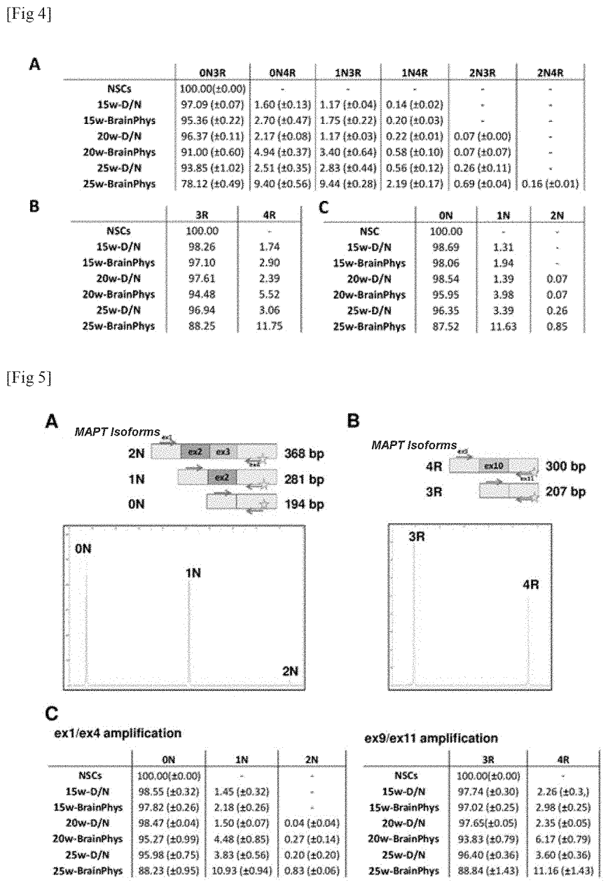

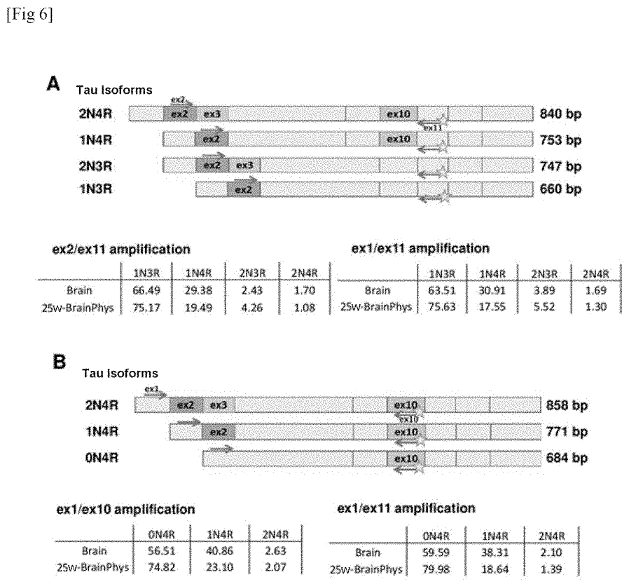

### DESCRIPTION OF THE DRAWINGS

[FIG. 1] shows a molecular characterization of iPSCs, NSCs and iPSC-derived neurons. RT-PCR analysis at 15, 20 and 25 weeks (15w, 20w, 25w), of the iPSCs (A), NSCs (B) and neurons differentiated at various differentiation points (C-F), using the pluripotency marker POU5F1 (A), the NSC marker OTX-1 (B), two markers specific for the neurons of the upper layer of the cortex: CALB1 (C) and RELN (D), GFAP as astrocyte marker (E) and the oligodendrocyte-specific marker CLDN11 (F). The cells were differentiated for 15, 20 or 25 weeks (w), in DMEM/F12—Neurobasal (D/N) or BrainPhys medium.

[FIG 2] shows the detection of the 6 adult isoforms of MAPT mRNA in brain extracts. (A) Schematic representation of the various MAPT isoforms analyzed. The expected sizes for each PCR product were calculated as a function of the position of the primers covering exons 1 and 11. (B) Each peak corresponds to a different MAPT isoform. The Y axis displays the fluorescence in arbitrary units and the X axis indicates the size in bp.

[FIG 3] shows the expression of the adult MAPT proteins and mRNA isoforms in iPSC-derived neurons. (A) Representation of the relative expression of the 6 MAPT isoforms in neurons induced from iPSCs after 15, 20 and 25 weeks of maturation by RT-qPCR analysis. The cells were differentiated for 15, 20 or 25 weeks (w), in DMEM/F12 Neurobasal (D/N) or BrainPhys medium. (B) Western Blot analysis of proteins extracted from 25-week neuronal capsules maintained in BrainPhys. The treatment with Lambda phosphatase reveals the presence of four Tau isoforms, corresponding to 1N4R, 1N3R, 0N4R and 0N3R. The 2N isoforms are not detectable.

[FIG 4] represents the quantification of the relative expression of the 6 adult isoforms of MAPT mRNA. The MAPT mRNA isoforms were quantified either individually (A), or grouped together as a function of the inclusion of exon 10 (B), or of the inclusion of exons 2 and 3 (C). (A) The data represent the mean standard deviation (SD) of at least two independent experiments.

[FIG 5] shows the detection and quantification of the adult isoforms of MAPT mRNA. Specific analyses of the relative proportion of the 0N, 1N and 2N isoforms (A), or of the 3R and 4R isoforms (B) in the NSCs or in the iPSC-derived neurons, by RT-qPCR. The neuronal cells were differentiated for 15, 20 or 25 weeks (w), in DMEM/F12—Neurobasal (D/N) or BrainPhys medium. For each analysis (A, B), a schematic representation of the various MAPT mRNA isoforms analyzed, the expected size of the PCR product calculated as a function of the position of the primers and a representative electropherogram are presented. The Y axis displays the fluorescence in arbitrary units and the X axis indicates the size in bp. (C) The quantitative values are indicated in the tables. The data represent the mean standard deviation (SD) of at least two independent experiments.

[FIG 6] shows the detection and quantification of the adult isoforms of MAPT mRNA after exclusion of the 0N3R transcripts. Specific analyses of the 1N3R, 1N4R, 2N3R and 2N4R transcripts (A) or of the 4R-Tau isoforms (B) in brain extracts or in neuronal capsules of 25 weeks, maintained in BrainPhys, by RT-qPCR. (A) The use of the primers ex2 and ex11 made it possible to amplify solely the 1N and 2N isoforms. (B) The use of the primers ex1 and ex10 allowed the specific amplification of the 4R isoforms. For each analysis (A, B), a schematic representation of the various MAPT isoforms analyzed and the expected size of the PCR product calculated as a function of the position of the primers are presented. The corresponding quantitative values are indicated in the tables on the left (A: ex2/ex11 amplification, B: ex1/ex10) amplification. The tables on the right (A, B: ex1/ex11) present the data obtained when the 6 MAPT mRNA isoforms were simultaneously amplified using ex1-ex11 (FIG. 4A) and the relative expression of the 1N3R, 1N4R, 2N3R and 2N4R isoforms calculated without including the 0N isoforms (A) or the relative expression of the 0N4R, 1N4R and 2N4R isoforms calculated without including the 3R isoforms (B).

### DETAILED DESCRIPTION OF THE INVENTION

The present invention relates to a method for culturing and producing post-mitotic neuronal cells expressing the 6 isoforms 2N4R, 1N4R, 0N4R, 2N3R, 1N3R, 0N3R of the Tau protein. This expression profile is characteristic of the adult neurons, and is not found in particular at the embryonic level. Thus far, the methods for the in vitro production of neuronal cells have not resulted in the production of neuronal cells expressing these 6 isoforms. It is to the credit of the inventors to have discovered and shown that it is possible to produce such neurons in vitro, at high throughput and in reasonable times (about a few tens of weeks), by combining a 3D cell culture with a differentiation medium.

The use of cellular microcompartments comprising an external hollow hydrogel shell encapsulating the cells and extracellular matrix is particularly advantageous and makes it possible, in the context of the present invention, to promote the cellular differentiation and the maturation of the neurons as far as the post-mitotic stage characteristic of adult neurons.

Cellular Microcompartment

The method according to the invention uses cellular microcompartments containing cells. Each cellular microcompartment comprises a hollow crosslinked hydrogel capsule, in which are housed cells embedded in an extracellular matrix.

In one embodiment, the hydrogel capsule contains a single assembly of cells. By single, it is meant that the capsule contains only one group of cells, which may be more or less cohesive. In particular, a single assembly of cells is understood to be a three-dimensional cellular structure in which each cell of said assembly is in physical contact with at least one other cell of said assembly.

Advantageously, the cells are self-organized into an assembly of cells positioned in a particular manner with respect to one another so as to create cell interactions and communications and to form a three-dimensional microstructure of interest. Each microcompartment thus comprises an external layer of hydrogel, or hydrogel capsule, containing an assembly of self-organized cells. The cells can multiply, become organized and/or differentiate within the hydrogel capsule.

In the context of the invention, “the external hydrogel layer” or “hydrogel shell” denotes a three-dimensional structure formed from a matrix of polymer chains swollen by a liquid, and preferentially water. Such an external hydrogel layer is obtained by crosslinking a hydrogel solution. Advantageously, the polymer(s) of the hydrogel solution are polymers that are crosslinkable when subjected to a stimulus, such as a temperature, a pH, ions, etc. Advantageously, the hydrogel solution used is biocompatible, in the sense that it is not toxic to the cells. The hydrogel layer advantageously allows the diffusion of dissolved gases (and in particular of oxygen and/or carbon dioxide), of nutrients, and of metabolic waste so as to allow survival, proliferation, differentiation, maturation of the cells and/or the production of molecules or of molecular assemblies of interest and/or the recapitulation of cellular behaviors of interest. The polymers of the hydrogel solution may be of natural or synthetic origin. For example, the hydrogel solution contains one or more polymers among sulfonate-based polymers, such as sodium polystyrene sulfonate, acrylate-based polymers, such as sodium polyacrylate, polyethylene glycol diacrylate, the gelatin methacrylate compound, polysaccharides, and in particular polysaccharides of bacterial origin, such as gellan gum, or of plant origin, such as pectin or alginate. In one embodiment, the hydrogel solution comprises at least alginate. Preferentially, the hydrogel solution comprises only alginate. In the context of the invention, the term “alginate” is intended to mean linear polysaccharides formed from β-D-mannuronate (M) and α-L-guluronate (G), and salts and derivatives thereof. Advantageously, the alginate is a sodium alginate, composed of more than 80% of G and less than 20% of M, with an average molecular mass of 100 to 400 kDa (for example: PRONOVA SLG100) and a total concentration comprised between 0.5% and 5% in density (weight/volume).

Preferentially, the cellular microcompartment is closed. It is the external hydrogel layer which gives the cellular microcompartment its size and its shape. The microcompartment may have any shape compatible with the encapsulation of cells.

Preferentially, the extracellular matrix layer forms a gel. The extracellular matrix layer comprises a mixture of proteins and of extracellular compounds required for cell culture, for example of pluripotent cells. Preferentially, the extracellular matrix comprises structural proteins, such as laminin 521, 511 or 421, entactin, vitronectin, laminins, collagen, and also growth factors, such as TGF-beta and/or EGF. In one embodiment, the extracellular matrix layer consists of, or contains, MATRIGEL and/or GELTREX.

According to the invention, the microcompartment can contain an extracellular matrix substitute in place of the extracellular matrix. An extracellular matrix substitute is understood to be a compound capable of promoting the attachment and/or the survival of cells by interacting with the membrane proteins and/or the extracellular signal transduction pathways. For example, such a substitute comprises biological polymers and fragments thereof, in particular proteins (laminins, vitronectins, fibronectins and collagens), nonsulfated glycosaminoglycans (hyaluronic acid) or sulfated glycosaminoglycans (chondroitin sulfate, dermatan sulfate, keratan sulfate, heparan sulfate), and synthetic polymers containing motifs derived from biological polymers or reproducing the properties thereof (RGD motif) and small molecules mimicking the attachment to a substrate (Rho-A kinase inhibitors such as Y-27632 or thiazovivin).

Any method for producing cellular microcompartments containing, inside a hydrogel capsule, extracellular matrix and cells can be used for carrying out the preparation method according to the invention. In particular, it is possible to prepare microcompartments by adapting the method and the microfluidic device described in Alessandri et al., 2016 (“A 3D printed microfluidic device for production of functionalized hydrogel microcapsules for culture and differentiation of human Neuronal Stem Cells (hNSC)”, Lab on a Chip, 2016, vol. 16, no. 9, p. 1593-1604).

Advantageously, the dimensions of the cellular microcompartment are controlled. In one embodiment, the cellular microcompartment according to the invention has a spherical shape. Preferentially, the diameter of such a microcompartment is between 10 μm and 1 mm, more preferentially between 75 and 750 μm, more preferentially between 100 and 500 μm, even more preferentially between 150 and 300 μm, +/−10%. In another embodiment, the cellular microcompartment according to the invention has an elongated shape. In particular, the microcompartment may have an ovoid or tubular shape. Advantageously, the smallest dimension of such an ovoid or tubular microcompartment is between 10 μm and 1 mm, more preferentially between 75 and 750 μm, more preferentially between 100 and 500 μm, even more preferentially between 150 and 300 μm, +/−10%. By “smallest dimension”, it is meant the double of the minimum distance between a point located on the external surface of the hydrogel layer and the center of the microcompartment.

In one particular embodiment, the thickness of the external hydrogel layer represents 5 to 40% of the radius of the microcompartment. The thickness of the extracellular matrix layer represents 5 to 80% of the radius of the microcompartment and is advantageously attached to the internal face of the hydrogel shell. This matrix layer may fill the space between the cells and the hydrogel shell. In the context of the invention, “the thickness” of a layer is the dimension of said layer extending radially relative to the center of the microcompartment.

Neuronal Fifferentiation Medium

According to the invention, post-mitotic neuronal cells expressing the 6 isoforms are obtained within a relatively short production time, and in particular less than 100 weeks, and more preferentially less than 50 weeks. For that, the microcompartments are maintained in a neuronal differentiation medium.

Such media are known to those skilled in the art. It is in particular possible to use medium referred to as N2B27 (500 ml DMEM/F12, 500 ml Neurobasal, 5 ml N2 medium supplement, 10 ml B27 medium supplement). Advantageously, such a medium is supplemented in a first phase (first 10 to 15 days, neural induction phase) with molecules that block the BMP2-linked signaling pathways (such as LDN-193189 or the Noggin protein) and the TGF-beta signaling pathway (such as SB-431542), and/or in an optional second phase with molecules that activate the EGF-1- and FGF-2-linked signaling pathways (neural stem cell phase amplification), and/or supplemented in a terminal differentiation phase with molecules that activate the neurotrophic signaling pathways (for example the BDNF transduction pathway) and molecules that block the Notch signal transduction pathway (for example the E compound or another gamma-secretase inhibitor).

In one particular embodiment, the cellular microcompartments, containing stem cells, optionally already differentiated into post-mitotic neuronal cells, are cultured in a particular neuronal differentiation medium, comprising at least one neuroactive inorganic salt, glycine, L-alanine and L-serine, said compounds each being present at a concentration which maintains the survival and the neural functionality of a neuronal cell. Those skilled in the art are able to adjust the concentrations so as to maintain the survival and the functionality of said cells.

In the context of the invention, the term “neuroactive” compound is intended to mean a compound, such as an inorganic salt, which significantly affects the neural activity of a cell (for example an electrophysiological activity). Such a neural activity can be substantially identical to the neural activity of a wild-type neuronal cell in its natural environment (in vivo).

In one embodiment, the at least one neuroactive inorganic salt is chosen from the group consisting of sodium chloride (NaCl), potassium chloride (KCl), calcium chloride (CaCl₂), magnesium sulfate (MgSO₄), magnesium chloride (MgCl₂), ferric nitrate (FeNO₃), zinc sulfate (ZnSO₄), cupric sulfate (CuSO₄), ferric sulfate (FeSO₄) and combinations thereof. In one embodiment, the neuronal differentiation medium comprises sodium chloride and at least one other neuroactive inorganic salt.

In certain embodiments, the sodium chloride concentration is comprised between 20 and 200 mM, preferentially between 70 and 150 mM, in particular at 120 mM+/−10%.

The concentration of the other neuroactive inorganic salts is preferentially comprised between 0.000001 and 10 mM, 0.000005 and 8 mM, 0.00001 and 6 mM, 0.00005 and 4 mM, 0.00005 and 2 mM, 0.0001 and 1 mM, 0.0005 and 0.5 mM, 0.001 and 0.05 mM, 0.01 and 0.05 mM.

The glycine concentration like the L-alanine concentration are advantageously comprised between 0.0001 and 0.05 mM. The L-serine concentration is advantageously comprised between 0.001 and 0.03 mM.

In one embodiment, the culture medium also comprises:

L-aspartic acid, preferentially at a concentration comprised between 0.00001 and 0.003 mM, and/or

L-glutamic acid, preferentially at a concentration comprised between 0.00001 and 0.02 mM, and/or

a pH-modulating agent, such as an inorganic salt.

For example, the modulating agent is an inorganic salt chosen from dibasic sodium phosphate, monobasic sodium phosphate and combinations thereof, said agent being advantageously at a concentration comprised between 0.001 and 1 mM. Alternatively, the modulating agent is sodium bicarbonate, advantageously at a concentration comprised between 1 and 35 mM.

Alternatively or additionally, the culture medium may comprise at least one of the following compounds: one or more amino acids, each amino acid being advantageously at a concentration comprised between 0.001 and 1 mM; one or more vitamins, each vitamin being advantageously at a concentration comprised between 0.00001 and 1 mM; a supplementary agent chosen from the group consisting of a protein, a neurotrophic factor, a steroid, a hormone, a fatty acid, a lipid, a vitamin, a mineral sulfate, an organic chemical compound, a monosaccharide, a nucleotide and combinations thereof; an energy substrate, such as sugar, sodium pyruvate and combinations thereof, preferentially at a concentration comprised between 0.1 and 5 mM; and a photosensitive agent, in particular riboflavin (B2) at a concentration comprised between 0.0001 and 0.0006 mM; and/or HEPES at a concentration comprised between 1 and 10 mM.

For example, the amino acid(s) are then advantageously chosen from L-alanyl-L-glutamine, L-arginine hydrochloride, L-asparagine-H20, cysteine-H2O hydrochloride, L-cystine 2HCl, L-histidine-H2O hydrochloride, L-isoleucine, L-leucine, L-lysine hydrochloride, L-methionine, L-phenylalanine, L-proline, L-threonine, L-tryptophan, L-tyrosine disodium salt dihydrate, L-valine, and combinations thereof.

For example, the vitamin(s) are chosen from the group consisting of choline chloride, D-calcium pantothenate (B5), folic acid (B9), i-Inositol, niacinamide (B3), pyridoxine hydrochloride, thiamine hydrochloride, vitamin B12 (cyanocobalamin), riboflavin (B2), and combinations thereof.

In one embodiment, the culture medium does not comprise serum. Alternatively or additionally, the osmolarity of the medium is comprised between 280 and 330 Osm/ml.

Examples of composition of neuronal differentiation medium that can be used for carrying out the method according to the invention are described in application WO2014/172580. The BRAINPHYS Neuronal Medium sold by the company STEMCELL Technologies is particularly suitable for use as a neuronal differentiation medium in the method of the invention.

Culture Steps

According to the invention, the microcompartments are cultured in the cell differentiation medium for a period comprised between 5 weeks and 100 weeks. Advantageously, the culture step in the differentiation medium is carried out for a period comprised between 5 and 50 weeks, preferentially between 10 and 50 weeks, between 20 and 50 weeks, between 25 and 50 weeks, between 20 and 40 weeks, between 25 and 40 weeks, between 20 and 30 weeks, between 25 and 30 weeks, more preferentially between 20 and 25 weeks, even more preferentially for approximately 24 weeks, 25 weeks, 26 weeks, 27 weeks, 28 weeks, 29 weeks, 30 weeks, in particular for 25 weeks+/−1 week. This is because the inventors have discovered that, after this period of time, all or some of the microcompartments contain neurons expressing the 6 isoforms of the Tau protein (2N4R, 1N4R, 0N4R, 2N3R, 1N3R, 0N3R).

According to the invention, the microcompartments comprising the post-mitotic neuronal cells can be obtained during a step of preculturing cellular microcompartments, each comprising a hollow hydrogel capsule surrounding a single clump of stem cells and extracellular matrix in a culture medium capable of inducing cell differentiation within said cellular microcompartments; the cells being advantageously organized in the form of a cyst inside the hydrogel capsules on conclusion of said preculturing step.

In the context of the invention, the term cyst denotes at least one layer of pluripotent cells organized around a central lumen. According to the invention, such a microcompartment thus comprises successively, around a central lumen, said layer of pluripotent cells, a layer of extracellular matrix, or of an extracellular matrix substitute, and the external hydrogel layer. The lumen is generated, at the time of formation of the cyst, by the cells which multiply and develop in layers on the extracellular matrix layer. Advantageously, the lumen contains a liquid and more particularly the culture medium.

The stem cells used for preparing the microcompartments are advantageously mammalian pluripotent stem cells, which may be human or nonhuman. A pluripotent stem cell, or pluripotent cell, is understood to be a cell which has the capacity to form all the tissues present in the whole organism of origin, without however being able to form a whole organism as such. In particular, the stem cells are chosen from induced pluripotent stem cells (IPS), embryonic stem cells (ES), transdifferentiated cells, and mixtures thereof. The transdifferentiated cells are understood to be cells for which differentiation into cells of interest is obtained without going through a pluripotency step. The cells go from their initial state (for example fibroblast or peripheral blood mononuclear cell) to a mature neuron terminal state by forcing the expression of a set of genes of the terminal state without ever transitioning through the pluripotent phenotype.

A method for preparing cysts is described in application WO2018/096277, as are culture media conducive to the organization of cells in the form of cysts within the microcompartments.

In another embodiment, neural progenitors are encapsulated in a hydrogel shell, said progenitors being advantageously capable of organizing in the form of cysts.

In general, once the cellular microcompartments contain predominantly post-mitotic neuronal cells, said compartments are placed in the neuronal differentiation medium. By predominantly, it is meant that the microcompartment comprises more than 50% by number of post-mitotic neuronal cells, preferentially more than 60%, 70%, 75%, 80%, 85%, 90%, 95%.

Advantageously, the neuronal differentiation step is carried out in a bioreactor, the cellular microcompartments being kept in suspension in an enclosure of said bioreactor containing the differentiation medium.

Since the cells are protected by the hydrogel shell from the stresses that can exist within the reactor, the flow through the bioreactor can be as strong as the hydrogel shell can withstand. In addition, the hydrogel shell of the cellular microcompartments preserves the cells against the mechanical stresses associated with collisions and prevents fusions of the multicellular elements (aggregates, microcarriers). The microcompartments are in suspension in the bioreactor, which allows access to the culture medium and diffusion in the homogeneous microcompartments, and also good convection.

The use of cellular microcompartments makes it possible to culture the cells in any type of bioreactor, equipped with a closed enclosure, and in particular in a bioreactor in batch feed mode, in fed batch feed mode or in continuous feed mode (perfusion). The use of these microcompartments is particularly advantageous in the case of culture in continuous feed mode. This is because, since the cells are protected by the hydrogel shell, it is possible to subject them to continuous flows, without any risk of weakening them.

In one embodiment, the bioreactor comprises an enclosure that can be hermetically closed. This makes it possible to control the atmosphere inside the bioreactor, and for example to culture the microcompartments under inert atmosphere.

The bioreactor can comprise an enclosure having a volume comprised between 1 ml and 10 000 l, preferentially between 5 ml and 10 000 l, between 10 ml and 10 000 l, between 100 ml and 10 000 l, between 200 ml and 10 000 l, between 500 ml and 10 000 l. In one embodiment, the enclosure has a volume of at least 1 ml. In one embodiment, the enclosure has a volume of at least 10 ml. In one embodiment, the enclosure has a volume of at least 100 ml. In one embodiment, the enclosure has a volume of at least 500 ml. In one embodiment, the enclosure has a volume of at least 1 l. In one embodiment, the enclosure has a volume of at least 10 l. In one embodiment, the enclosure has a volume of 100 l, or more.

Those skilled in the art will know how to adjust the number of microcompartments and the volume of the bioreactor as required.

Preferentially, the neural differentiation step is carried out under sterile conditions in order to avoid any contamination by microorganisms. For example, the enclosure of the bioreactor is closed so as to prevent contaminations, but allows gas exchanges with the outside.

In general, the use of a closed enclosure allows a fine control of the culture environment, without any risk of disruption by the outside environment. It is moreover easy to obtain sterile products. This also allows a better volumetric efficiency.

On conclusion of the neuronal differentiation step, it is possible to recover all or some of the cellular microcompartments, in order to recover post-mitotic neurons expressing the 6 isoforms of the Tau protein that are contained in said microcompartments. The cells can be easily recovered, by simple hydrolysis and/or dissolution of the external hydrogel layer.

Neuronal Cells and Applications

The method according to the invention makes it possible to obtain post-mitotic neuronal cells in which the 6 isoforms 2N4R, 1N4R, 0N4R, 2N3R, 1N3R, 0N3R of the Tau protein are expressed.

Advantageously, the 6 isoforms are proportions substantially identical to the proportions present in wild-type adult neuronal cells.

In one embodiment, the post-mitotic neuronal cells recovered from the cellular microcompartments on conclusion of the differentiation step exhibit a ratio of expression of the 3R and 4R isoforms comprised between 1/3 and 3, more preferentially comprised between 1/2 and 2, even more preferentially comprised between 3/4 and 4/3, ideally at 10% of an equimolar ratio. The 2N, 1N and 0N isoforms advantageously represent, respectively, more than 3%, more than 17% and less than 90% of the total isoforms, preferentially, respectively, more than 5%, more than 26% and less than 50%, even more preferentially, respectively, more than 8%, more than 45% and less than 45%, ideally, respectively, 9%, 54%, and 37%.

In one embodiment, the post-mitotic neuronal cells recovered from the cellular microcompartments on conclusion of the differentiation step comprise the 6 isoforms in the proportions below, relative to the total number of the 6 isoforms: between 0.1 and 0.9% of the 2N4R isoform, in particular 0.16% or 0.9%, between 0.5 and 1% of the 2N3R isoform, in particular 0.69% or 1%, between 2 and 18% of the 1N4R isoform, in particular 2.19% or 17.6%, between 8 and 23% of the 0N4R isoform, in particular 9.4% or 22.4%, between 8 and 23% of the 1N3R isoform, in particular 9.4% or 27.5%, and between 30 and 80% of the 0N3R isoform, in particular 78.16% or 30.6%.

Such post-mitotic neuronal cells can be used both for research purposes and for diagnosis or treatment. These cells, exhibiting a Tau protein expression profile substantially identical to that of wild-type adult neuronal cells, are particularly suitable for screening for therapeutic molecules which target neurodegenerative diseases and/or which modify the physiopathology of neurons, in particular in human beings.

### EXAMPLES

Materials and Methods

1. iPSC Line and Encapsulation

The BC-1 line (WT XY, passages 15-25, MTI-Globalstem, Gaithersburg, Md.) was maintained in the absence of feeder cells. The culture plates were covered with a Matrigel matrix for 2 hours at 37° C. (Corning, N.Y., 1/100, diluted in a DMEM medium). The BC-1 colonies were dissociated using ReLeSR (Stemcell Technologies, Vancouver, Canada) and then cultivated in mTESR1 (STEMCELL Technologies) supplemented with 1% of penicillin/streptomycin (Invitrogen, Carlsbad, Calif.). The cultures were fed daily and passaged every 5 to 7 days.

The encapsulation procedure used is that described in Alessandri et al., 2016.

2. Production of Mature Cortical Neurons

Neural induction was carried out in a 1:1 mixture of DMEM/F12 and of Neurobasal supplemented with B27 and N2 (Thermo Fischer Scientific Inc., Waltham, Mass.), 1 μM LDN-193189 (Sigma Aldrich, St. Louis, Mo.) and 10 μM SB431542 (Tocris Bioscience, Bristol, UK). The medium was changed every day for 8 days. The differentiation of the neural stem cells was then carried out using the DMEM/F12:Neurobasal (1:1) mixture supplemented with B27 and N2, or the BRAINPHYS medium supplemented with N2-A and SM1 (Stemcell Technologies). The two media were supplemented with 10 ng/ml of BDNF and GDNF (Cell Guidance Systems Ltd., Cambridge, United Kingdom), 10 nM of Compound E (Abcam, Cambridge, United Kingdom) and 10 nM of trichostatin A (Abcam). Half the medium was changed every day until the specified maturation period.

3. RNA Extraction

The sedimented microcompartments (spheres) were rinsed once in 1× PBS (Thermo Fischer Scientific Inc.) and then incubated with Gentle Cell Dissociation Reagent (Stemcell Technologies) for 5 minutes in order to break up the alginate capsule. After two 1× PBS washes, the total RNA of the cells was extracted using the Nucleospin RNA XS kit (Macherey-Nagel GmbH and Co KG, Düren, Germany). Validation experiments were carried out on the total RNA extracted from normal adult human cerebral cortex (BioChain Institute Inc., Newark, Calif.).

4. Neuronal Maturation Analysis by RT-PCR

A total of 250 ng of RNA was reverse transcribed using the RETROscript reverse transcription kit (Thermo Fischer Scientific Inc.) with oligo(dT) primers, in accordance with the manufacturer's instructions for the two-step RT-PCR procedure. The PCR reactions were carried out using 1 μl of cDNA and the appropriate primers (FIG. 7). The PCR products were separated by electrophoresis on a 1.5% agarose gel.

5. Analysis of the Adult MAPT Isoforms by Fluorescent RT-PCR

Reverse transcription was carried out on 70 ng of RNA, using the Verso cDNA kit (Thermo Fischer Scientific Inc.) and oligo(dT) primers. The primers used in this study are listed in Table 1 below. The relative proportion of the MAPTisoforms was then analyzed by fluorescent PCR, using an unlabeled forward primer and a 6-FAM-labeled reverse primer. The PCR products were analyzed on a Genetic Analyzer 3500 automatic sequencer (Applied Biosystems), and the electropherograms were analyzed with GeneMapper Software 5.

TABLE 1

| Genes and | |
|---|---|
| oligonucleotides | |
| POU5F1 F (SEQ ID NO: | 5′-CTTGGGCTCGAGAAGGATGT-3′ |
| 1): | |
| POU5F1 R (SEQ ID NO: | 5′-GGAAAGGGACCGAGGAGTAC-3′ |
| 2): | |
| OTX-1 F (SEQ ID NO: 3): | 5′-CAAGACTCGCTACCCTGACA-3′ |
| OTX-1 R (SEQ ID NO: 4): | 5′-AGTAGGAAGAGGAGGGCGTA-3′ |
| CALB1 F (SEQ ID NO: 5): | 5′-TGGCTCACGTATTACCCACA-3′ |
| CALB1 R (SEQ ID NO: 6): | 5′-AGATCCGTTCGGTACAGCTT-3′ |
| RELN F (SEQ ID NO: 7): | 5′-ACTTTCCTCCTAGCGCTGTT-3′ |
| RELN R (SEQ ID NO: 8): | 5′-ATAATCGCGCCACACTGTTC-3′ |
| GFAP F (SEQ ID NO: 9): | 5′-AGAAGCTCCAGGATGAAACC-3′ |
| GFAP R (SEQ ID NO: 10): | 5′-AGCGACTCAATCTTCCTCTC-3′ |
| GLDN11 F (SEQ ID NO: | 5′-CTGGTGGACATCCTCATCCT-3′ |
| 11): | |
| GLDN11 R (SEQ ID NO: | 5′-CCAGCAGAATGAGCAAAACA-3′ |
| 12): | |
| Tau exon 1 (SEQ ID NO: | 5′-GCCAGGAGTTCGAAGTGATG-3′ |
| 13): | |
| Tau exon 2 (SEQ ID NO: | 5′-GAGGACGGATCTGAGGAACC-3′ |
| 14): | |
| Tau exons 4/5 (SEQ ID | 5′-CATGCGAGCTTGGGTCAC-3′ |
| NO: 15): | |
| Tau exon 9 (SEQ ID NO: | 5′-CCCAAGTCGCCGTCTTCC-3′ |
| 16): | |
| Tau exon 10 (SEQ ID NO: | 5′-ACTTGGACTGGACGTTGCTA-3′ |
| 17): | |
| Tau exon 11 (SEQ ID NO: | 5′-TGGTTTATGATGGATGTTGCC |
| 18): | T-3′ |

6. Protein Extraction and Dephosphorylation

The spheres were rinsed in 1× PBS (Thermo Fischer Scientific Inc.) and then incubated with Gentle Cell Dissociation Reagent (Stemcell Technologies) for 5 minutes in order to break up the alginate capsule. After two washes in 1× PBS, the spheres were homogenized in a PIERCE RIPA buffer (Thermo Fisher Scientific Inc.) supplemented with a cocktail of protease inhibitors (Sigma-Aldrich), using TissueLyserLT (Qiagen, Hilden, Germany) (rapid stirring (50 Hz, 2 min), 1.5 ml microtubes containing two 2 mm stainless steel balls). The samples were then centrifuged and the lysates were collected. After 30 min on ice and centrifugation (11 300×g, 20 min, 4° C.), the supernatant containing the soluble proteins was collected. Thirty μg of total proteins were then treated with 80 units of lambda protein phosphatase (New England Biolabs, Ipswich, Mass.) for 3 hat 37° C. in a final volume of 50 μl.

7. Western Blotting

The proteins were analyzed by means of the Western Blotting technique as described previously (Pons et al., 2017). Briefly, the proteins of the samples that had been dephosphorylated were separated by electrophoresis on a 10% SDS-PAGE gel, in parallel with 1 μl of a reference sample containing the 6 Tau protein isoforms produced in vitro (Sigma Aldrich), then transferred onto a nitrocellulose membrane. The membranes were incubated with the primary antibody: Polyclonal Anti-human Tau (Dako Denmark A/S, Glostrup, Denmark) (1:50 000) then revealed using chemiluminescent reagents (ECL Clarity, Bio-Rad Laboratories).

Results

1. Development of Cortical Neurons and of Glial Lineages on Matrigel Starting from iPSC in Alginate Capsules

In this study, iPSCs originating from healthy donors were used. After dissociation, the iPSC cells were encapsulated inside Matrigel-lined alginate capsules as described previously for the neural stem cells (NSC) in Alessandri et al., 2016. The capsules were first maintained in a mTESR medium in order to allow colonies to emerge inside the capsules. Neural induction of the iPSCs was then carried out by double inhibition of SMAD until the NSCs reached 100% confluence in the capsules (Feyeux et al., 2012). The neural induction efficiency was confirmed by evaluation of POU5F1 and OTX-1 mRNA expression by RT-PCR. As expected, the POU5F1 pluripotency gene was strongly expressed in the iPSCs and virtually undetected in the NSCs (FIG. 1A). Furthermore, the NSC marker OTX-1 was specifically overexpressed in the NSCs compared to the noninduced iPSCs (FIG. 1B). Once the NSCs had reached confluence, they were then differentiated in cortical neurons. Two cell culture media were tested: a commonly used medium (DMEM/F12—Neurobasal 1:1, D/N) and also the BRAINPHYS medium which was designed to promote maturation and synaptic function of iPSC-derived neurons (Bardy et al., 2015). Because prior studies have shown that cortical neurogenesis from iPSCs follows the same time course in vitro as the cortical development of mammals and extends at least up to day 90 in culture (Espuny-Camacho et al., 2013; Kirwan et al., 2015; Shi et al., 2012), it was arbitrarily decided to characterize the identity of the iPSC-derived human cortical neurons in the experimental procedure after 15, 20 or 25 weeks of culture. The expression of CALB1 (Calbindin 1) and RELN (Reelin), two markers specific for the cortical neurons of the upper layer, was evaluated. The cortical neurons of the upper layer are generated at the final stages of cortical neurogenesis. CALB1 is expressed in cortical layers II and III of the human cerebral cortex and RELN is expressed in cortical layer I. As shown in FIGS. 1C and 1D, CALB1 and RELN mRNA expression was detected in the iPSC-derived neurons from 15 weeks of culture regardless of the medium used. The expression of the two genes was maintained up to 25 weeks. As expected, CALB1 and RELN were not detected at a significant level in the iPSCs and the NSCs.

The presence of astrocytes and of myelinating oligodendrocytes in the cultures was also studied, by evaluating respectively the expression of GFAP (Glial fibrillary acidic protein) and CLDN11 (Claudin11) mRNAs. GFAP expression was detected from 15 weeks of culture under the two experimental conditions (FIG. 1E). On the other hand, while the expression of CLDN11 was easily detected in capsules cultured in the BRAINPHYS medium from the 15th week of culture, its expression remained absent in cells cultured in the standard medium, even after 25 weeks of differentiation (FIG. 1F). Once again, as expected, none of these genes is expressed at the iPSC and NSC stages. On the whole, these results indicate that the neurons derived from iPSC inside the Matrigel-lined alginate capsules were able to differentiate into cortical neurons, including the cortical neurons of the upper layer which are generated at the final stages of cortical neurogenesis. It is important to note the presence of oligodendrocytes specifically in the cultures maintained in BRAINPHYS, underlining that the BRAINPHYS neuronal medium improved neuronal maturation compared to the commonly used D/N medium.

2. Development of a Quantitative Method for Measuring the 6 Adult MAPT mRNA Isoforms: Validation in the Human Cerebral Cortex

Currently, analyses of the expression of the adult MAPT mRNA isoforms are mainly based solely on the quantification of the inclusion of exon 10, in order to determine the 3R/4R ratio, or the inclusion of exons 2 and 3, in order to evaluate the proportion of the 0N, 1N or 2N isoforms. In the present study, a method allowing the simultaneous analysis of the 6 adult MAPT mRNA isoforms individually in a single PCR reaction in order to examine their relative proportion during neuronal maturation was developed. For this purpose, a new test was developed, based on the amplification of the MAPT transcripts using fluorescence-labeled primers. A set of primers covering exons 1 and 11 (FIG. 2A), which should allow the amplification of the 6 MAPT isoforms, was identified. In order to validate this test, RT-PCR experiments on mRNAs extracted from adult human cerebral cortex were carried out. The analysis of the electropherograms corresponding to the products amplified by PCR revealed a profile in the form of 6 peaks corresponding to the expected sizes for the 6 adultMAPTmRNA isoforms (FIG. 2B). The relative proportion of the various isoforms was then calculated from the height of the peak. The 0N3R and 1N3R isoforms were the most expressed (30.6 and 27.5%), followed by the 0N4R and 1N4R isoforms (22.4 and 17.6%), while the 2N3R and 2N4R isoforms represented only 1 and 0.9% of all the isoforms.

These data also made it possible to determine the relative proportion of the 0N (53%), 1N (45.1%) and 2N (1.9%) isoforms and of the 3R (59.1%) and 4R (40.9%) isoforms. It is important to note that similar quantitative data were obtained during the independent analysis of the inclusion of exons 2/3 and of the inclusion of exon 10 (FIGS. 5A, B): the 0N and 1N isoforms proved to be the most expressed (52.9% and 45% respectively), whereas the 2N isoforms represented only 2%. With regard to the splicing of exon 10, the relative quantification of the peaks indicates that the ratio is slightly in favor of the 3R isoforms, with 59.9% of 3R-Tau compared with 40.1% of 4R-Tau. These data are perfectly consistent with the studies previously published and therefore validate this new test.

3. BRAINPHYS Promotes the Expression of the 6 Adult MAPT mRNA Isoforms in iPSC-Derived Neurons Cultured inside Matrigel-Lined Alginate Capsules

After having validated the test on adult brain extracts, the method was used to evaluate the expression of the adult MAPT isoforms in iPSC-derived neuronal cultures maintained in D/N or in a BRAINPHYS medium, over a maturation period of 25 weeks. In accordance with prior studies, the NSC analysis revealed a single peak, corresponding to the predicted size of the 0N3R isoform of MAPT (FIG. 3A and FIG. 4A). Isoforms containing exon 2 (the 1N3R and 1N4R isoforms) and exon 10 (the 0N4R and 1N4R isoforms) were detected from 15 weeks of maturation. It is interesting to note that the cultures maintained in BRAINPHYS exhibited higher levels of expression of the 1N3R, 0N4R and 1N4R isoforms than the D/N cultures. After 20 weeks of maturation, a significant increase in the amount of these species was observed, with the appearance of low levels of expression of the 2N3R isoform under the two experimental conditions. At the final point of the study (25 weeks), the levels of expression of these adult MAPT mRNA isoforms continued to increase. Even more important, the 2N4R isoform was now detectable in the cultures maintained in BRAINPHYS. Quantitative analysis of the relative proportions of the MAPT mRNA isoforms in cultures maintained using BRAINPHYS for 25 weeks revealed that the 0N4R and 1N3R isoforms each represented 9.4% of all the isoforms present, while the 1N4R isoform represented 2.19%. The 2N3R and 2N4R isoforms corresponded respectively to 0.69% and 0.16%. The 4R-Tau isoforms therefore constituted 11.75% of the MAPT transcripts (FIG. 4B), while the 1N and 2N isoforms represented respectively 11.63% and 0.85% (FIG. 4C). As with the brain extracts, these results were validated by independent evaluations of the splicing of exon 10 or of exons 2/3 (FIG. 5C).

The experimental procedure allowed the development of neurons expressing the 6 adult MAPT mRNA transcripts, but the 0N3R isoform remained mainly expressed after 25 weeks of maturation (˜78%). In order to rule out the possibility that the 0N3R isoforms can “trap” the primers and therefore decrease the efficiency of the PCR compared to the less well expressed transcripts, the expression of the MAPT isoforms was analyzed independently of the 0N3R transcripts. To do this, two sets of primers (FIG. 6) were produced: the first covering exons 2 and 11 in order to accurately analyze the 1N3R, 1N4R, 2N3R and 2N4R transcriptions, and the second covering exons 1 and 10 in order to specifically amplify the 4R-Tau isoforms. The analysis of brain extracts and also of the cultures maintained in BRAINPHYS for 25 weeks confirmed the relative proportions of the various MAPT isoforms, thus validating the test.

As a whole, these results showed that the neurons maintained in BRAINPHYS expressed the 6 adult MAPT transcripts after 25 weeks of maturation, including the 2N3R and 2N4R isoforms. It is important to note that when the iPSC-derived cultures were preserved in the D/N medium, only 5 isoforms were expressed. The 2N4R isoform was undetectable. Furthermore, at each study point, the mRNA levels for each isoform were systemically lower than those observed with the BRAINPHYS medium. These data are perfectly consistent with the beneficial role of the BRAINPHYS medium on the maturation state of neurons. It is interesting to note that, in accordance with what has been demonstrated during the development of the human brain (Hefti et al., 2018), a change in the expression of exon 2 and exon 10 was first detected. The inclusion of exon 3 occurs later.

In order to validate the efficiency of the translation of the adult MAPT transcripts in the experimental model, the production of Tau protein isoforms was then evaluated by Western Blotting in spheres maintained in BRAINPHYS for 25 weeks. The proteins were optionally dephosphorylated using lambda phosphatase and separated by electrophoresis next to a recombinant Tau protein ladder containing the 6 isoforms. As shown in FIG. 3B, the Tau proteins migrate in the form of multiple bands between 40 and 60 kDa, as a result (i) of the presence of multiple isoforms and (ii) of the phosphorylation state of the proteins. The phosphatase treatment led to a shift of the Tau proteins toward lower molecular weights. In accordance with the mRNA analysis, it was noted that the 0N3R-Tau isoform was predominantly detected in the 25-week capsules. However, significant amounts of 0N4R, 1N3R and 1N4R isoforms were also detected. On the other hand, it was not possible to observe the 2N Tau isoforms. Since these isoforms represent less than 1% of all the Tau species present in as mRNA, it is possible that their concentrations are lower than the limit of detection in the analysis.

### CONCLUSIONS

These results show that iPSC-derived neurons cultured inside Matrigel-lined alginate capsules exhibited mature cortical cell culture specifications. Even more important, by means of a new test which allows the qualitative and quantitative analysis of all the adult MAPT mRNA isoforms individually, it was demonstrated that the neurons maintained in BRAINPHYS expressed the 6 adult MAPT mRNA transcripts after 25 weeks of maturation, making this model very suitable for the modeling of Tau pathologies and for therapeutic purposes.

1-11. (canceled)

12. A method for the in vitro production of mammalian neurons expressing 6 isoforms of the Tau protein, the six isoforms being 2N4R, 1N4R, 0N4R, 2N3R, 1N3R, and 0N3R, the method comprising a step of neuronal differentiation, according to which cellular microcompartments are cultured for a period comprised between 5 weeks and 100 weeks, each comprising a hollow hydrogel capsule surrounding post-mitotic neuronal cells and an extracellular matrix, said neuronal differentiation step being carried out in a bioreactor, the cellular microcompartments being kept in suspension in an enclosure of said bioreactor containing a neuronal differentiation medium.

13. The method for the in vitro production of mammalian neurons according to claim 12, wherein the neuronal differentiation medium comprises at least one neuroactive inorganic salt, glycine, L-alanine and L-serine.

14. The method for the in vitro production of mammalian neurons according to claim 13, wherein the neuroactive inorganic salt is chosen from the group consisting of sodium chloride, potassium chloride, calcium chloride, magnesium sulfate, magnesium chloride, ferric nitrate, zinc sulfate, cupric sulfate, ferric sulfate, and combinations thereof.

15. The method for the in vitro production of mammalian neurons according to claim 12, wherein the neuronal differentiation step is carried out for a period comprised between 5 and 50 weeks, between 10 and 50 weeks, between 20 and 25 weeks, or for 25 weeks+/−1 week.

16. The method for the in vitro production of mammalian neurons according to claim 12, wherein the neural differentiation step is carried out under sterile conditions, the enclosure of the bioreactor being a closed enclosure.

17. The method for the in vitro production of mammalian neurons according to claim 12, the method comprising a preculturing step according to which the microcompartments of post-mitotic neuronal cells are obtained by culturing cellular microcompartments each comprising a hollow hydrogel capsule surrounding a single clump of stem cells, with the exclusion of human embryonic stem cells, and extracellular matrix in a culture medium capable of inducing cell differentiation within said cellular microcompartments.

18. The method for the in vitro production of mammalian neurons according to claim 17, wherein the cells are organized in the form of a cyst inside the hydrogel capsules on conclusion of said preculturing step.

19. The method for the in vitro production of mammalian neurons according to claim 17, wherein the stem cells are pluripotent stem cells selected from the group consisting of induced pluripotent stem cells (IPS), embryonic stem cells (ES), with the exclusion of human embryonic stem cells, transdifferentiated cells, and mixtures thereof.

20. The method for the in vitro production of mammalian neurons according to claim 12, the method comprising a subsequent step according to which, on conclusion of the neuronal differentiation step, post-mitotic neurons expressing the 6 isoforms of the Tau protein are recovered from the cellular microcompartments.

21. The method for the in vitro production of mammalian neurons according to claim 12, wherein the cellular microcompartments have a diameter comprised between 10 μm and 1 mm, between 75 and 750 μm, between 100 and 500 μm, or between 150 and 300 μm, +/−10%.

22. Non-natural post-mitotic neuronal cells, which can be obtained by the method according to claim 12, in which 6 isoforms of the Tau protein are expressed, the 6 isoforms of the Tau protein being 2N4R, 1N4R, 0N4R, 2N3R, 1N3R, and 0N3R.

23. The non-natural post-mitotic neuronal cells according to claim 22, said cells exhibiting a ratio of expression of the 3R and 4R isoforms between 1/3 and 3, between 1/2 and 2, or between 3/4 and 4/3.

24. The non-natural post-mitotic neuronal cells according to claim 22, wherein the 3R and 4R isoforms are at 10% of equimolar ratio.

25. The non-natural post-mitotic neuronal cells according to claim 22, wherein the 2N, 1N and 0N isoforms represent, respectively, more than 3%, more than 17% and less than 90% of the total isoforms, more than 5%, more than 26% and less than 50%, more than 8%, more than 45% and less than 45%, or respectively, 9%, 54%, and 37%.
